(19) United States
(12) Patent Application Publication
(10) Pub. No.: US 2026/0090781 A1
(43) Pub. Date: Apr. 2, 2026

(54) METHOD AND SYSTEM FOR PROVIDING PARAMETER DATA FOR A CONTROL SCAN OF A COMPUTED TOMOGRAPHY SCANNER

(71) Applicant: Siemens Healthineers AG, Forchheim (DE)

(72) Inventors: Matthias BAER-BECK, Spardorf (DE); Christian HOFMANN, Erlangen (DE); Johan SUNNEGAARDH, Adelsdorf (DE)

(73) Assignee: Siemens Healthineers AG, Forchheim (DE)

(21) Appl. No.: 19/330,884

(22) Filed: Sep. 17, 2025

(30) Foreign Application Priority Data
Oct. 2, 2024 (EP) 24204141.6

(51) Int. Cl. A61B 6/00; A61B 6/03; A61B 6/40

(52) U.S. Cl. CPC A61B 6/545; A61B 6/032; A61B 6/4007

(57) ABSTRACT

A computer-implemented method for providing parameter data for performing at least one control scan with a computed tomography scanner, comprises receiving object path data, the object path data at least comprising geometric information about a planned movement path of an object; receiving predefined control scan data, the predefined control scan data at least comprising information about a position and an orientation of at least one scan plane of at least one planned control scan for controlling the movement path of the object; receiving an evaluation model configured to provide parameter data for performing the at least one control scan at least depending on the object path data and the predefined control scan data; and applying the evaluation model and providing the parameter data for performing the at least one control scan.

### CROSS-REFERENCE TO RELATED APPLICATION(S)

The present application claims priority under 35 U.S.C. § 119 to European Patent Application No. 24204141.6, filed Oct. 2, 2024, the entire contents of which are incorporated herein by reference.

### FIELD

The present disclosure relates to a computer-implemented method for providing parameter data for carrying out at least one control scan with a computed tomography scanner arrangement, a system for providing parameter data for carrying out at least one control scan with a computed tomography scanner arrangement and a respective computer program element.

### RELATED ART

For some medical procedures, like vertebroplasties, kyphoplasties, biopsies, ablations, drainages or endoleak repairs, it is necessary to bring objects, for example a needle-shaped medical equipment into a patient's body. For such procedures, it is common use, that the examination is supported with repeated online computed tomography imaging in order to control a current position of the object in the patient. Due to the material of these objects, which are typically made of metal, and their geometrical shape, image artifacts can usually not be avoided when the objects are introduced into the patient's body.

It is a common clinical workflow to insert the object, e.g. so-called needles, along paths that are completely within the scan plane of the computed tomography scanner. The reason is that those paths are typically more ergonomic, easier to plan and easier to follow during the medical procedure. But this comes at the drawback of increased image artifacts in the reconstructed control scan images. Typical artifacts are dark streaks along the object path which extend beyond the physical position of the object. Those artifacts are caused by the physical effects of beam-hardening and scatter and the image quality degrading impact of those effects is strongest for the projections which are aligned parallel to the object path. Here, at least for detector pixels that are in the shadow of the object, x-rays are attenuated along the whole object which results in a quite low primary signal, i.e. the signal without artificial contributions, e.g., from scattered radiation, behind the object. In the extreme case, when the dose of the computed tomography scans which are done to control the object position during the medical procedure is reduced to a low level, it may happen that parts of the object disappear in the computed tomography image. The reason for this is that in some of the projection data that are acquired during the computed tomography scan, the measurements that are in the shadow of the object are below the detection level of the x-ray detector and result in a zero-measurement. In these cases, the signal from the object is superimposed, for example by scatter artifacts, quantum noise or electronics noise, so that it cannot be restored in the reconstructed computed tomography image.

### SUMMARY

In this context, it has become apparent that there is a further need to provide a method and/or a system allowing an improved reconstruction of computed tomography images of a computed tomography scanner arrangement (also referred to as a computed tomography scanner), in particular allowing an improved reconstruction of computed tomography images of control scans of a computed tomography scanner arrangement.

The present disclosure provides a method and/or a system providing an improved reconstruction of computed tomography images of a computed tomography scanner arrangement, such as providing an improved reconstruction of computed tomography images of control scans of a computed tomography scanner arrangement.

These and other objects, which become apparent upon reading the following description, are solved by the subject matters of the independent claims. The dependent claims refer to preferred embodiments of the present disclosure.

### BRIEF DESCRIPTION OF THE DRAWINGS

In the following, the present disclosure is further described with reference to the enclosed figures:

FIG. 1 illustrates a flow diagram of a computer-implemented method for providing parameter data for carrying out at least one control scan with a computed tomography scanner arrangement according to an embodiment of the present disclosure;

FIG. 2 illustrates a system for providing parameter data for carrying out at least one control scan with a computed tomography scanner arrangement according to an embodiment of the present disclosure;

FIG. 3 is a schematic side view of a computed tomography scanner arrangement according to an embodiment of the present disclosure; and

FIG. 4 is a schematic top view of a computed tomography scanner arrangement according to an embodiment of the present disclosure.

### DETAILED DESCRIPTION

In one aspect of the present disclosure, a computer-implemented method for providing parameter data for carrying out at least one control scan with a computed tomography scanner arrangement is disclosed, comprising:
receiving object path data, at least comprising geometric information about a planned movement path of an object;
receiving predefined control scan data, at least comprising information about the position and orientation of at least one scan plane of at least one planned control scan for controlling the movement path of the object;
receiving an evaluation model configured to provide parameter data for performing the at least one control scan at least depending on the received object path data and the received control scan data;
applying the evaluation model, in particular, to at least the object path data and the predefined control scan data, and providing parameter data for performing the at least one control scan, wherein at least the object path data and the predefined control scan data are input data of the evaluation model.

In other words, the present disclosure proposes to evaluate, by the evaluation model, the object path and, in particular, the predefined position and orientation of the scan plane for the at least one planned control scan, whether there is a risk/probability of image artifacts when the planned control scan is performed. If the evaluation model determines that there is no risk or that the risk is below a predefined threshold value, the already predefined control scan data can be used to perform the control scan without modification by the parameter data. If the evolution model determines that there is a risk or that the risk is above a specified threshold value, adjustments/modifications to the predefined control scan data may be made via the parameter data trying to reduce the risk of image artifacts in the control scan. These adjustments/modifications may relate, for example, to the radiation dose settings and/or the orientation of adjustable moving parts of the computed tomography scanner arrangement adopting the scan plane of the control scan.

Therefore, the present disclosure proposed a method which could evaluated potential vanishing object/needle issues in advance before a control scan is performed. Moreover, if a potential vanishing object/needle issue is evaluated, it is possible to automatically adapt the parameters/settings of a control scans in advance, so that the vanishing abject/needle artifact may not show up in a reconstructed computed tomography scanner image.

The term “parameter data” is to be understood broadly in this context and includes, in particular, all data relating to the specific execution of a control scan. For example, parameters for the dosage of X-ray radiation, parameters relating to the positioning and alignment of movable parts of the computed tomography scanner arrangement and the like.

The term “object” is also to be understood broadly in this context and includes all medical devices that can be introduced into a patient and whose position can be checked by computed tomography scanner images. For example, an object can be so-called needles, such as biopsy needles or the like.

The term “object path data” is also to be understood broadly in the present case and may include data that relates to the geometry of the object and a planned movement of the object through a patient. For example, the object path data may include the geometry data of an object and a path that this object is to be moved through a patient's body from a starting point to an end point.

The term “predefined control scan data” is also to be understood broadly in the present case and may include information about the position and orientation of at least one scan plane of at least one planned control scan. The predefined control scan data may also include other predefined parameters, such as predefined X-ray doses, movements and/or trajectories of the computed tomography scanner arrangement during the control scan and the like.

The term “evaluation model” is also to be understood broadly in this context and includes simple fall group distinctions, i.e. predefined parameter data output for certain object path data and predefined control scan data. However, more complex evaluation models may also be used. In an example, the evaluation model may comprise at least one trained algorithm, wherein the term “trained algorithm” or “trained evaluation model” as used herein is to be understood broadly in the present case. The algorithm may be a machine learning algorithm. The algorithm may comprise decision trees, naive bayes classifications, nearest neighbors, neural networks, convolutional or recurrent neural networks, transformers, generative adversarial networks, support vector machines, linear regression, logistic regression, random forest, gradient boosting algorithms and/or a diffusion model. Such an algorithm, in particular machine learning algorithm, is termed “intelligent” because it is capable of being “trained.”

The algorithm may be trained using records of training data. A record of training data comprises training input data and corresponding training output data. In the context of the present disclosure, training data may, for example be object path data, predefined control scan data and at least one parameter data output for performing the at least one control scan. The training output data of a record of training data is the result that is expected to be produced by the algorithm when being given the training input data of the same record of training data as input. The deviation between this expected result and the actual result produced by the algorithm is observed and rated via a “loss function”. This loss function may be used as feedback for adjusting the parameters of the internal processing chain of the algorithm. For example, the parameters may be adjusted with the optimization goal of minimizing the values of the loss function that result when all training input data are fed into the algorithm and the outcome is compared with the corresponding training output data. The result of this training is that given a relatively small number of records of training data as “ground truth”, the algorithm is enabled to perform its job well for a number of records of input data that is higher by many orders of magnitude. Notably, the evaluation model may comprise several or even a large number of different evaluation routines/sub-models. It is also possible to use mixed evaluation routines that combine different evaluation approaches.

In an embodiment of the computer-implemented method, the evaluation model may be configured to calculate and evaluate the intersection length of the radiation cone with the planned object path for a projection of the at least one control scan. In this respect, the evaluation model may be configured to provide parameter data based on a comparison of the intersection length and at least one predefined threshold value. For example, if there is a projection where the intersection length of the radiation cone with the planned object/needle path exceeds a certain predefined threshold, for such a projection a certain risk of vanishing object/needle artifacts may be evaluated. If an intersection length matches a needle path, a high risk of vanishing object/needle artifacts may be evaluated

In an embodiment of the computer-implemented method, the method may further comprise:
receiving dose data, comprising planned dose settings for the at least one planned control scan;
receiving tissue data, comprising tissue information at least in the area of the planned movement path of the object;
wherein the evaluation model is further configured to provide parameter data for performing the at least one control scan further depending on the dose data and the tissue data; and wherein the dose data and the tissue data are further input data of the evaluation model.

Via further evaluating the planned/predetermined dose settings of the planned control scan and the tissue data/information in the area of the planned control scan, the risk of vanishing object/needle artifacts may be evaluated with higher accuracy. In this respect, at least tissue data may be received relating to the tissue through which the scan passes, so that the damping of the radiation by the tissue may be considered by the evaluation model. The tissue data may be provided, for example, by a previously performed scan, e.g. a so-called planning scan. Using the tissue data of the patient, the total, i.e. via tissue and object/needle, attenuation for the vulnerable projections identified in the above-mentioned step may be determined. Based on the control scan dose settings, e.g. tube current and voltage, the signal that the detector would detect in the shadow of the object/needle for the projections may be determined. If this signal is above the detection threshold of the computed tomography scanner arrangement or a predefined detection threshold, it may be assumed that the projections in question may not produce relevant image artifacts. If the signal in the object/needle shadow is below the detection threshold or a predefined detection threshold, it may be assumed that the projections may produce image artifacts, e.g. vanishing object/needle artifacts, and a change in the control scan setup via the parameter data may be triggered.

In an embodiment of the computer-implemented method, the parameter data may comprise tilting, alignment and/or positioning information/data with respect to a position, alignment and/or tilt of a gantry arrangement of the computed tomography scanner arrangement. A tilting mechanism of a computed tomography scanner arrangement may be used to tilt the scan plane of the control scan so that the intersection of the object path with the radiation cone may be reduced. Thereby, the gantry's angles of inclination and rotation may be optimized so that the angle between the resulting scan plane and the object/needle path is as close as possible to 90°. Using such a geometry setting for the scan ensures that the cross-section of the object/needle path with the scan plane is as small as possible and that only a fraction of the projections of a full 360° scan are needed to reconstruct and determine a correct position of the object in the reconstructed computed tomography image for small, highly attenuating objects without interesting internal structure.

In an embodiment of the computer-implemented method, the parameter data may comprise dose setting data causing the computed tomography scanner arrangement to use a harder spectrum for the control scan. For example, 150 kV with tin filtration instead of 120 kV. Notably, computed tomography scans typically use a tube current-based dose modulation to optimize patient dose and image quality. When changing the tube voltage for the entire scan, the tube current profile may be configured in such a way that the total dose to the patient may remain constant.

In an embodiment of the computer-implemented method, the parameter data may comprise dose setting data causing the computed tomography scanner arrangement to modify a tube current modulation profile increasing the tube current for at least one projection of the object/needle path, in particular for those projections for which a risk of image artifacts was identified. In particular, via changing the tube current modulation profile, the tube current may be increased for projections that are parallel or within a predefined threshold to the object/needle path. The total patient dose may be kept constant by reducing the tube current for the remaining projections that are considered to have no or a low risk for image artifacts, e.g. which are not parallel to the object/needle path. Alternatively, the total patient dose may also be increased, which can be justified by the improved image quality of the control scans.

In an embodiment of the computer-implemented method, the parameter data may comprise dose setting data causing the computed tomography scanner arrangement to modify a tube voltage using a harder spectrum for at least one projection of the object/needle path, in particular for those projections for which a risk of image artifacts was identified. In particular, via changing the tube voltage, a harder spectrum may be used for projections that are parallel or within a predefined threshold to the object/needle path. For example, the spectrum may be modified to 150 kV with tin filtration instead of 120 kV.

In an embodiment of the computer-implemented method, the parameter data may comprise:
tilting, alignment and/or positioning information/data with respect to a position, alignment and/or tilt of a gantry arrangement of the computed tomography scanner arrangement;
dose setting data causing the computed tomography scanner arrangement to use a higher/harder tube voltage for the control scan;
dose setting data causing the computed tomography scanner arrangement to modify a tube current modulation profile increasing the tube current for at least one projection of the object/needle path, in particular for those projections for which a risk of image artifacts was identified; and/or
dose setting data causing the computed tomography scanner arrangement to modify a tube voltage using a harder spectrum for at least one projection of the object/needle path, in particular for those projections for which a risk of image artifacts was identified.

In an embodiment of the computer-implemented method, the parameter data may comprise scan data causing the computed tomography scanner arrangement to turn on the tube only for predetermined projections and/or to turn on only tubes according to a predetermined schedule. Today, standard spiral or sequence scans are usually used for computed tomography guided procedures to perform the control scans for object/needle positioning. In order to reduce the overall dose to a patient, these images are typically acquired at a reduced dose, but still a full set of raw data is acquired. Full here means that a standard computed tomography scanner image may be reconstructed from the acquired raw data, i.e. the raw data set contains at least projections within an angular interval of 180°+fan angle of the computed tomography scanner. In this respect, the total dose can be reduced, for example, by turning on the tube only for a fraction of the predetermined projections, depending on the geometry of the planned needle path.

In case a so called static computed tomography scanner arrangement with distributed radiation sources is used, the overall radiation dose may be further reduced. Static computed tomography scanner arrangement means that the scanner is not assembled according to the geometry principle of the third generation. A static computed tomography scanner arrangement has a ring detector, e.g. a full 360° detector ring, and a source ring, wherein the source ring comprises several small X-ray tubes or a distributed source, e.g. based on field-effect emitter cathodes, with a constant increment. During data collection, these tubes can be switched on and off independently and, in any order, allowing that for a control scan only predetermined X-ray tubes or distributed radiation sources are turned on corresponding to the needle/object path.

In an embodiment of the computer-implemented method, the method may further comprise:
providing control data for controlling a computed tomography scanner arrangement based on the parameter data for the at least one control scan.

By providing control data based on the parameter data, the computed tomography scanner arrangement may be fully automated to adapt to the planned control scan.

A further aspect of the present disclosure relates to a system for providing parameter data for carrying out at least one control scan with a computed tomography scanner arrangement, comprising:
a processing circuitry;
a storage medium; and
a data interface;
wherein the storage medium comprises a computer program that comprises instructions which when the program is executed, cause the processing circuitry to carry out the above-explained computer-implemented method;
wherein the data interface is configured to receive input data for the evaluation model including the object path data and the predefined control scan data.

In an embodiment of the system, the system may comprise a computed tomography scanner, preferably comprising a tilting/movement mechanism configured to change the gantry's angle of inclination and rotation.

In an embodiment of the system, the system may comprise a computed tomography scanner with distributed radiation sources. A static computed tomography scanner arrangement has a ring detector, e.g. a full 360° detector ring, and a radiation source ring, wherein the radiation source ring comprises several small X-ray tubes or a distributed radiation source, e.g. based on field-effect emitter cathodes, with a constant increment.

A further aspect of the present disclosure relates to a computer program element with instructions, which, when executed on computing devices of a computing environment, is configured to carry out the steps of the above-described computer-implemented, in particular, in an above-described system. The computer program element might be stored on a computing unit of a computing device, which might also be part of an embodiment. This computing unit may be configured to perform or induce performing of the steps of the method described above. Moreover, it may be configured to operate the components of the above-described system. The computing unit can be configured to operate automatically and/or to execute the orders of a user. The computing unit may include a data processor. A computer program may be loaded into a working memory of a data processor. The data processor may thus be equipped to carry out the method according to one of the preceding embodiments. This exemplary embodiment of the present disclosure covers both, a computer program that right from the beginning uses the present disclosure and computer program that via an update turns an existing program into a program that uses the present disclosure. Moreover, the computer program element might be able to provide all necessary steps to fulfill the procedure of an exemplary embodiment of the method as described above.

According to a further exemplary embodiment of the present disclosure, a computer readable medium, such as a CD-ROM, USB stick, a downloadable executable or the like, is presented wherein the computer readable medium has a computer program element stored on it, which computer program element is described by the preceding section. A computer program may be stored and/or distributed on a suitable medium, such as an optical storage medium or a solid-state medium supplied together with or as part of other hardware, but may also be distributed in other forms, such as via the internet or other wired or wireless telecommunication systems. However, the computer program may also be presented over a network like the World Wide Web and can be downloaded into the working memory of a data processor from such a network. According to a further exemplary embodiment of the present disclosure, a medium for making a computer program element available for downloading is provided, which computer program element is arranged to perform a method according to one of the previously described embodiments of the present disclosure.

A further aspect of the present disclosure relates to a use of
object path data, at least comprising geometric information about a planned movement path of an object;
predefined control scan data, at least comprising information about the position and orientation of at least one scan plane of at least one planned control scan for controlling the movement path of the object; and/or
an evaluation model configured to provide parameter data for performing the at least one control scan at least depending on the received object path data and the received control scan data;
in an above-described computer-implemented method for providing parameter data for carrying out at least one control scan with a computed tomography scanner arrangement.

This and embodiments described herein relate to the methods, the systems, the apparatuses, the computer program element, the computer-readable storage medium, the use lined out above and vice versa. Advantageously, the benefits provided by any of the embodiments and examples equally apply to all other embodiments and examples and vice versa.

The term “control data” as used herein is to be understood broadly in the present case and comprises any data structure/information structure that is suitable for controlling a robot component, for example in digital form and/or in analog form.

The term “data” as used herein is to be understood broadly in the present case and represents any kind of data. Data may be single numbers/numerical values, a plurality of a numbers/numerical values, a plurality of a numbers/numerical values being arranged within a list, 2 dimensional maps or 3 dimensional maps, but are not limited thereto.

The term “providing” and the term “receiving” used herein are to be understood broadly in the present case and could be related to any of providing, receiving, querying, measuring, calculating, determining, transmitting of data, but are not limited thereto. Data may be provided to a user via a user interface, in particular, by depicting/showing the data on a display. Data may be provided to another device by transmitting the data to the other device. Data may be received from another device. The data may by queried from the other device, measured by the other device, calculated by the other device, determined by the other device and/or transmitted by the other device.

In the following particularly preferred embodiments are disclosed, which may be combined with the above-disclosed methods, systems, apparatuses, devices and/or use cases. The embodiments described herein may be combined unless specifically noted otherwise. Features and advantages described with respect to one aspect of the disclosure may be applied to other aspects of the disclosure.

The following embodiments are mere examples for implementing the method, the system, disclosed herein and shall not be considered limiting.

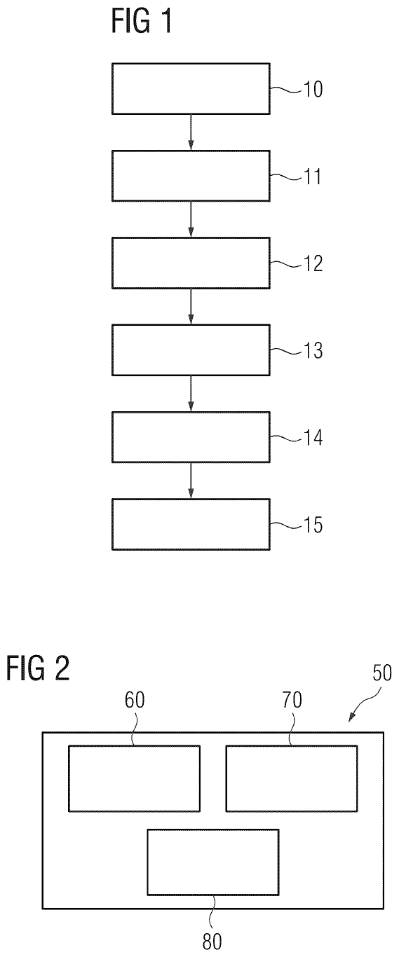

FIG. 1 illustrates a flow diagram of a computer-implemented method for providing parameter data for carrying out at least one control scan with a computed tomography scanner arrangement according to an embodiment of the present disclosure.

In a first step 10, object/needle path data, at least comprising geometric information about a planned movement path of an object are received. Such object/needle path data may be provided by a previously performed scan, e.g. a so-called planning scan. For example, the object path data may include the geometry data of an object and a path that this object is to be moved through a patient's body from a starting point to an end point.

In a second step 11, predefined control scan data, at least comprising information about the position and orientation of at least one scan plane of at least one planned control scan for controlling the movement path of the object are received. The predefined control scan data may be entered in a previous step automatically, partially automatically and/or manually. For example, the predefined control scan data may be predefined by default program settings of a computed tomography scanner arrangement.

In a further step 12, an evaluation model configured to provide parameter data for performing the at least one control scan at least depending on the received object path data and the received control scan data is received. The evaluation model may comprise predefined parameter data output for certain object path data and predefined control scan data. However, more complex evaluation models may also be used. For example, the evaluation model may comprise at least one trained algorithm, wherein the term “trained algorithm” or “trained evaluation model”. In an example of the present disclosure, the evaluation model is configured to calculate and evaluate the intersection length of the radiation cone with the planned object path for a projection of the at least one control scan. In this respect, the evaluation model may be configured to provide parameter data based on a comparison of the intersection length and at least one predefined threshold value. For example, if there is a projection where the intersection length of the radiation cone with the planned object/needle path exceeds a certain predefined threshold, for such a projection a certain risk of vanishing object/needle artifacts may be evaluated. If an intersection length matches a needle path, a high risk of vanishing object/needle artifacts may be evaluated.

In a further step 13, the evaluation model is applied and the parameter data for performing the at least one control scan is provided, wherein at least the object path data and the predefined control scan data are input data of the evaluation model.

In an example of the present disclosure, the parameter data may comprise:
tilting, alignment and/or positioning information/data with respect to a position, alignment and/or tilt of a gantry arrangement of the computed tomography scanner arrangement;
dose setting data causing the computed tomography scanner arrangement to use a higher/harder tube voltage for the control scan;
dose setting data causing the computed tomography scanner arrangement to modify a tube current modulation profile increasing the tube current for at least one projection of the object/needle path, in particular for those projections for which a risk of image artifacts was identified; and/or
dose setting data causing the computed tomography scanner arrangement to modify a tube voltage using a harder spectrum for at least one projection of the object/needle path, in particular for those projections for which a risk of image artifacts was identified.

Thus, the present disclosure proposes to evaluate, by the evaluation model, the object path and, in particular, the predefined position and orientation of the scan plane for the at least one planned control scan, whether there is a risk/probability of image artifacts when the planned control scan is performed. If the evaluation model determines that there is no risk or that the risk is below a predefined threshold value, the already predefined control scan data can be used to perform the control scan without modification by the parameter data. If the evolution model determines that there is a risk or that the risk is above a specified threshold value, adjustments/modifications to the predefined control scan data may be made via the parameter data trying to reduce the risk of image artifacts in the control scan. These adjustments/modifications may relate, for example, to the radiation dose settings and/or the orientation of adjustable moving parts of the computed tomography scanner arrangement adopting the scan plane of the control scan.

In an optional further step 14, dose data comprising planned dose settings for the at least one planned control scan are received. In a further optional step 15, tissue data comprising tissue information at least in the area of the planned movement path of the object are received. Such tissue data of a patient may be provided by a previously performed scan, e.g. a so-called planning scan.

FIG. 2 illustrates a system 50 for providing parameter data for carrying out at least one control scan with a computed tomography scanner arrangement, comprising: a processing circuitry 60, a storage medium 70, and a data interface 80. The storage medium 70 comprises a computer program with instructions which when the program is executed, cause the processing circuitry 60 to carry out the computer-implemented method for providing parameter data for carrying out at least one control scan with a computed tomography scanner arrangement according to an embodiment of the present disclosure. The data interface 80 is configured to receive at least input data for the evaluation model including the object path data and the predefined control scan data.

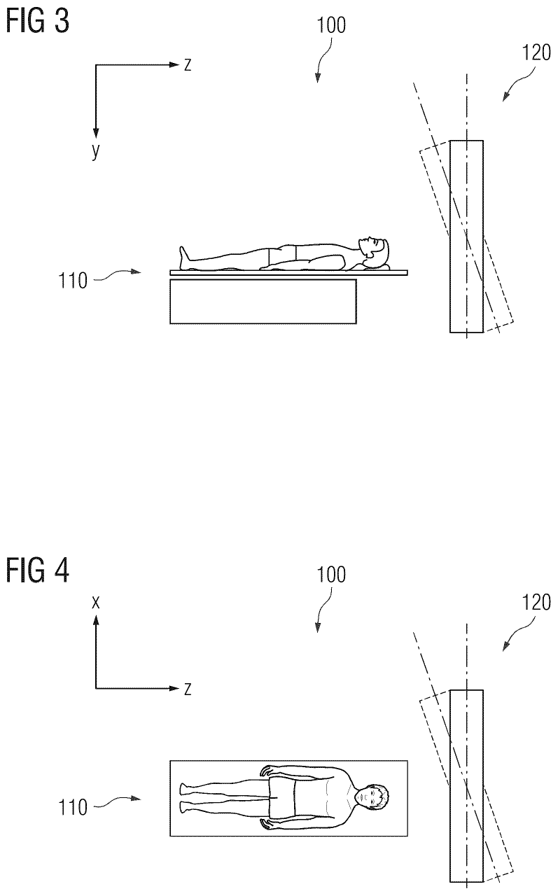

In FIGS. 3 and 4, an example of a computed tomography scanner arrangement 100 is illustrated. The computed tomography scanner arrangement 100 comprises a patient table 110, on which a patient may be placed, and a gantry 120 comprising the radiation source(s) and the detector. The computed tomography scanner arrangement 100 may comprise a tilting/rotating mechanism which may be used to tilt and/or rotate the gantry and thus the scan plane of the control scan so that the intersection of the object path with the radiation cone may be reduced. Thereby, the gantry's angle of inclination (cf. FIG. 3) relative to the patient table 110 around a horizontal tilt axis, the horizontal tilt axis being perpendicular to a horizontal longitudinal axis of the patient table 110, and/or the gantry's angle of rotation (cf. FIG. 4) relative to the patient table 110 around a vertical rotation axis may be optimized, for example, so that the angle between the resulting scan plane and the object/needle path is as close as possible to 90°. Using such a geometry setting for the scan may ensure that the cross-section of the object/needle path with the scan plane is as small as possible and that only a fraction of the projections of a full 360° scan are needed to reconstruct and determine a correct position of the object in the reconstructed computed tomography image for small, highly attenuating objects without interesting internal structure.

As indicated in FIGS. 3 and 4, usually, the spatial coding is based on an X-Y-Z coordinate system. The Z-axis is usually defined as an axis of symmetry of the gantry of the computed tomography scanner arrangement through an opening of the gantry. In the usual setup of a computed tomography scanner arrangement, the Z-axis is oriented horizontally and runs centrally through the opening of the gantry. As indicated in the FIGS. 3 and 4, the patient is usually brought into the opening parallel to the Z-axis on the patient table 110. Together with the Z-coordinate axis, an X-coordinate axis and a Y-coordinate axis span a space, wherein the coordinate axes are preferably orthogonal to one another and the X-coordinate axis is horizontal and the Y-coordinate axis is vertical.

The present disclosure has been described in conjunction with a preferred embodiment as examples as well. However, other variations can be understood and effected by those persons skilled in the art and practicing the claimed invention, from the studies of the drawings, this disclosure and the claims. Notably, in particular, the any steps presented can be performed in any order, i.e. the present invention is not limited to a specific order of these steps. Moreover, it is also not required that the different steps are performed at a certain place or at one node of a distributed system, i.e. each of the steps may be performed at a different node using different equipment/data processing units.

As used herein “determining” also includes “estimating, calculating, initiating or causing to determine”, “generating” also includes “initiating or causing to generate” and “providing” also includes “initiating or causing to determine, generate, select, send, query or receive”.

In the claims as well as in the description the word “comprising” does not exclude other elements or steps and the indefinite article “a” or “an” does not exclude a plurality. A single element or other unit may fulfill the functions of several entities or items recited in the claims. The mere fact that certain measures are recited in the mutual different dependent claims does not indicate that a combination of these measures cannot be used in an advantageous implementation. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for providing parameter data for carrying out at least one control scan with a computed tomography scanner, the method comprising:
receiving object path data, the object path data at least comprising geometric information about a planned movement path of an object;
receiving predefined control scan data, the predefined control scan data at least comprising information about a position and an orientation of at least one scan plane of at least one planned control scan for controlling the movement path of the object;
receiving an evaluation model configured to provide parameter data for performing the at least one control scan at least depending on the object path data and the predefined control scan data; and
applying the evaluation model and providing the parameter data for performing the at least one control scan, wherein at least the object path data and the predefined control scan data are input data of the evaluation model.

2. The computer-implemented method of claim 1, wherein the evaluation model is configured to calculate and evaluate an intersection length of a radiation cone with the planned movement path of the object for a projection of the at least one control scan.

3. The computer-implemented method of claim 2, wherein the evaluation model is configured to provide the parameter data based on a comparison of the intersection length and at least one predefined threshold value.

4. The computer-implemented method of claim 1, the method further comprising:
receiving dose data, the dose data comprising planned dose settings for the at least one planned control scan; and
receiving tissue data, the tissue data comprising tissue information at least in an area of the planned movement path of the object,
wherein the evaluation model is further configured to provide the parameter data for performing the at least one control scan further depending on the dose data and the tissue data, and
the dose data and the tissue data are further input data of the evaluation model.

5. The computer-implemented method of claim 1, the parameter data comprise at least one of
tilting,
alignment, or
positioning information with respect to at least one of a position, an alignment, or a tilt of a gantry of the computed tomography scanner.

6. The computer-implemented method of claim 1, wherein the parameter data comprise dose setting data causing the computed tomography scanner to use a relatively higher tube voltage for the control scan.

7. The computer-implemented method of claim 1, wherein the parameter data comprise dose setting data causing the computed tomography scanner to modify a tube current modulation profile increasing the tube current for at least one projection of an object path.

8. The computer-implemented method of claim 1, wherein the parameter data comprise scan data causing the computed tomography scanner to at least one of turn a tube on only for predetermined projections or turn on only tubes according to a predetermined schedule.

9. The computer-implemented method of claim 1, further comprising:
providing control data for controlling the computed tomography scanner based on the parameter data for the at least one control scan.

10. A system for providing parameter data for carrying out at least one control scan with a computed tomography scanner, the system comprising:
processing circuitry;
a storage medium; and
a data interface,
wherein the storage medium comprises instructions, when executed by the processing circuitry, cause the system to perform the method of claim 1, and
the data interface is configured to receive input data for the evaluation model including the object path data and the predefined control scan data.

11. The system of claim 10, further comprising:
the computed tomography scanner, the computer tomography scanner comprising a movement mechanism configured to change at least one of an angle of inclination or a rotation of a gantry of the computer tomography scanner.

12. The system of claim 10, further comprising:
the computed tomography scanner, the computed tomography scanner including a ring detector and a radiation source ring including multiple X-ray tubes or a distributed radiation source.

13. A non-transitory computer-readable medium including instructions, when executed on computing devices of a computing environment, cause the computing environment to perform the method of claim 1.

14. The computer-implemented method of claim 2, the method further comprising:
receiving dose data, the dose data comprising planned dose settings for the at least one planned control scan; and
receiving tissue data, the tissue data comprising tissue information at least in an area of the planned movement path of the object,
wherein the evaluation model is further configured to provide the parameter data for performing the at least one control scan further depending on the dose data and the tissue data, and
the dose data and the tissue data are further input data of the evaluation model.

15. The computer-implemented method of claim 14, the parameter data comprises at least one of
tilting,
alignment, or
positioning information with respect to at least one of a position, an alignment, or a tilt of a gantry of the computed tomography scanner.

16. The computer-implemented method of claim 15, wherein the parameter data comprise dose setting data causing the computed tomography scanner to use a relatively higher tube voltage for the control scan.

17. The computer-implemented method of claim 15, wherein the parameter data comprise dose setting data causing the computed tomography scanner to modify a tube current modulation profile increasing the tube current for at least one projection of an object path.

18. The computer-implemented method of claim 15, wherein the parameter data comprise scan data causing the computed tomography scanner to at least one of turn a tube on only for predetermined projections or turn on only tubes according to a predetermined schedule.

19. The computer-implemented method of claim 15, further comprising:
providing control data for controlling the computed tomography scanner based on the parameter data for the at least one control scan.
